(19) United States
(12) Patent Application Publication
(10) Pub. No.: US 2005/0142660 A1
(43) Pub. Date: Jun. 30, 2005

(54) Conditioned medium for culturing Schwann cells

(75) Inventors: Wan-Shiun Lou, Taichung City (TW); Yu-Hua Chen, Taichung City (TW); Pei-Ching Chang, Yuanlin Township (TW); Hsin-Hsin Shen, Taipei City (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsinchu (TW)

(21) Appl. No.: 10/990,465

(22) Filed: Nov. 18, 2004

(30) Foreign Application Priority Data
Dec. 30, 2003 (TW) 092137525

(51) Int. Cl. C12N 5/08

(52) U.S. Cl. 435/368; 435/404

(57) ABSTRACT

The invention provides a conditioned medium that enhances the survival and/or proliferation of Schwann cells in cell culture, and a cell line useful for production of such medium. The cell line used in the invention is a differentiated NTera2/D1 (NT2) cell line.

### BACKGROUND OF THE INVENTION

1. Field of the Invention

The invention relates to a medium and, more particularly, to a conditional medium for culturing Schwann cells.

2. Description of Related Art

2.1. Schwann Cells

Schwann cells (SCs) are the principal support cells in the peripheral nervous system. The cells originate from the neural crest during early embryonic development and migrate with the extending axons of the nerve into the periphery. During this phase, Schwann cells undergo rapid proliferation to produce an adequate number of cells to accommodate the growing axons. Subsequently, Schwann cells become terminally differentiated by ensheathing or myelinating the axons and then remain quiescent during adult life. However, Schwann cell proliferation can be stimulated under pathological conditions and plays a crucial role in nerve regeneration following injury. When a peripheral nerve is transected, Schwann cells at the site of the injury begin to demyelinate and reenter the cell cycle (Bunge, R. P. ,Curr. Opin. Neurobiol. 3: 805-809,1993). The proliferating Schwann cells produce neurotropic factors and extracellular matrix proteins to guide or facilitate the regrowth of the transected axons and finally complete the process of regeneration by remyelinating the regenerated axons.

The remarkable capacity of Schwann cells to promote nerve fiber regeneration, in both the peripheral and central nervous system, has been demonstrated by peripheral nerve graft (Paino, C. L. and Bunge, M. B., Exp. Neurol. 114: 254-257,1991) and the implantation of guidance channels impregnated with Schwann cells (V Guenard et al., J. Neuroscience 12: 3310-3320, 1992 and Paino C L et al., J. Neurocytol. 23:433-452,1994). A cellular prosthesis containing human Schwann cells has been proposed for clinical applications such as transplantation to the site of spinal cord injury to influence the regeneration of central axons and to repair complex peripheral nerve injuries containing lengthy gaps (Levi A. D. O et al., J. Neuroscience 14(3):1309-1319,1994). The clinical success of these procedures, which use autologous Schwann cells, depends on the ability to expand in vitro a pure Schwann cell population starting from material in a small biopsy.

Several reports have described techniques for culturing Schwann cells. See Carola Meier et al., J Neurosci 19(10):3847-3859,1999; Cheng, L. et al., Mol. Cell. Neurosci. 12:141-156, 1998; Daniel E. Syroid et al., J Neurosci 19(6):2059-2068,1999; Lobsiger C S, et al. Glia. 30(3):290-300,2000; Parkinson D B et al. Mol Cell Neurosci. 20(1):154-67,2002; U.S. Pat. No. 5,594,114; U.S. Pat. No. 5,714,385; U.S. Pat. No. 5, 721,139; U.S. Pat. No. 5,849,585; U.S. Pat. No. 6,417,160; Verdú E. et al., J Neurosci Methods 99(1-2): 111-117,2000; and WO 00/09139. However, these culturing method described above have limited effects for the survival and proliferation of Schwann cells.

There is a need, therefore, to provide an effective culture method that enhance the survival and proliferation of Schwann cells in cell culture.

2.2. Conditioned Medium (CM)

Culture medium compositions typically include essential amino acids, salts, vitamins, minerals, trace metals, sugars, lipids and nucleosides. Cell culture medium attempts to supply the components necessary to meet the nutritional needs required to grow cells in a controlled, artificial and in vitro environment. Nutrient formulations, pH, and osmolarity vary in accordance with parameters such as cell type, cell density, and the culture system employed. Many cell culture medium formulations are documented in the literature and a number of media are commercially available. Once the culture medium is incubated with cells, it is known to those skilled in the art as “conditioned medium”. The conditioned medium contains many of the original components of the medium, as well as a variety of cellular metabolites and secreted proteins, including, for example, biologically active growth factors, inflammatory mediators and other extracellular proteins. Thus, conditioned media are often used for the cultivation of particularly fastidious cells and cell lines, such as embryonic stem cells, because these substances described above may promote the growth of new cells and may help them to “take”.

### SUMMARY OF THE INVENTION

The invention provides media that enhance the survival and/or proliferation of Schwann cells in cell culture, and cell lines useful for production of such media.

One aspect of the invention is a method for preparing a conditioned medium suitable for culturing Schwann cells in a growth environment, comprising conditioning medium by culturing cells in the medium, and then harvesting the conditioned medium.

The cells used for conditioning the medium are taken from a cell line that can produce useful substances capable of enhancing the survival and/or proliferation of Schwann cells in cell culture. The cell line used in the invention is a differentiated NTera2/D1 (NT2) cell line, derived from a human teratocarinoma. Exemplary cell lines are obtained by differentiating NTera2/D1 (NT2) cells in vitro.

Another aspect of the invention is a method for culturing Schwann cells, comprising steps of providing a conditioned medium, and culturing Schwann cells in a growth environment containing the conditioned medium.

Another aspect of the invention is a conditioned medium for enhancing the survival and/or proliferation of Schwann cells in a growth environment, the conditioned medium is prepared according to a method of the invention.

These and other aspects of the invention will become more apparent from the following detailed description when taken in conjunction with the accompanying drawings.

### BRIEF DESCRIPTION OF THE DRAWINGS

FIG. 1A shows the growth condition of Schwann cells cultured in NT2 conditioned medium/SC basic medium at the fifteenth day;

FIG. 1B shows the growth condition of Schwann cells cultured in SC basic medium at the fifteenth day;

FIG. 2A shows the morphology of Schwann cells by an Optical microscope; and

FIG. 2B shows the green fluorescence expression of S-100 protein in Schwann cells cultured in vitro.

### DETAILED DESCRIPTION OF THE PREFERRED EMBODIMENT

The invention is directed to media that enhance the survival and/or proliferation of Schwann cells in cell culture, and the Schwann cells are benefit in clinical use for nerve repair.

In one aspect, the invention provides a method for preparing a conditioned medium which comprising: (a) providing a culture medium, and cells; (b) culturing cells in a culture medium, thereby conditioning the medium; and (c) harvesting the conditioned medium from the culture.

The cells used for conditioning the medium can produce useful substances capable of enhancing the survival and/or proliferation of Schwann cells in cell culture, as described earlier. The cell line used in the invention is a differentiated NTera2/D1 (NT2) cell line, derived from a human teratocarcinoma. Exemplary cell lines are obtained by differentiating NTera2/D1 (NT2) cells in vitro. Thus, the method of the invention can further include an additional step of differentiating NTera2/D1 (NT2) cells.

Methods for differentiating NTera2/D1 (NT2) cells are well known in the art (see for example U.S. Pat. No. 5,175,103, and Andrews P W et al., Differentiation 43: 131-138, 1990). The differentiating method includes the steps of: providing undifferentiated NTera2/D1 (NT2) cells, and incubating the cells in a culture medium containing differentiation agents such as hexamethylene bisacetamide (HMBA), retinoic acid (RA) or bromodeoxyuridine (BrdUrd). In certain preferred embodiments, the differentiation agent is RA. Determination that the NTera2/D1 (NT2) cells are differentiated can be done by morphological criteria.

In general, after 1 week treated with 10 μM of RA, the morphology of NTera2/D1 (NT2) cells was changed, which was characterized by outgrowing dendrites and axons; after 14-21 days of RA treatment, the cells irreversibly loose their dividing capacity. Continued culturing for a period time in medium, results in a culture consisting of neuron-like cells lying on top of less differentiated flat “pan-cake-like” cells(Lee, V. M.-Y and Andrews, P. W., J. Neurosci. 6(2):514-521,1986; Pleasure, S. J.et al., J Neurosci 12(5):1802-1815,1992).

The culture medium used for conditioned contains several different formula, depending in part on the types of cells that used. The medium must be able to culture the cell line used for medium conditioning at least. Preferably, the medium can also culture the Schwann cells after it was conditioned.

The preparation of culture medium for culturing Ntera2/D1 (NT2) cells are well known in the art and can be referred to the article by Andrews, P. W. Dev. Biol. 103: 285-293, 1984. In certain preferred embodiments, the culture medium is DMEM/F-12 (Dulbecco's modified Eagle's medium/Ham's nutrient mixture F-12)

The culture medium is then seeded with the differentiated Ntera2/D1 (NT2) cells in an environment for a sufficient period that allows the cells to secret useful substances into the medium, to enhance the survival and/or proliferation of Schwann cells in cell culture. Typically, to produce qualified conditioned medium for Schwann cells, the differentiated NT2 cells are cultured in the medium for 24 h at 37° C. at least 10 days thereby conditioning the medium efficiently. However, the conditioning period can be adjusted upwards or downwards, determining empirically what constitutes an adequate period.

The conditioned medium produced by the method described above can be used directly without any further process; preferably, the medium is filtrated after harvesting or before use. The filtration means is unlimited in the invention; preferably, the filtration means is a filter with 0.22 μm pore size.

In another aspect, the invention provides a method for culturing Schwann cells which comprising: culturing the Schwann cells in a growth environment that contains a conditioned medium produced by a method comprising: (a) providing a culture medium, and cells; (b) culturing the cells in the culture medium, thereby conditioning the medium; and then (c) harvesting the conditioned medium from the culture; wherein the cells used to condition the medium are differentiated Ntera2/D1 (NT2) cells.

In a particularly preferred embodiment, media are used effectively after dilution (for example, conditioned medium: diluent medium is 1:1).

Thus, the method of the invention further includes an additional step of diluting the conditioned medium. The diluting method includes the steps of: providing a conditioned medium and a diluent medium; and mixing the conditioned medium with the diluent medium. In certain preferred embodiments, the diluent medium is Schwann cell culture medium (SC medium).

The SC medium can have any different formula and their preparation methods are well known in the art (see, for example, Daniel E. Syroid et al., J. Neurosci.19:2059-2068,1999; Gerburg Keihoff et al., J. Neurosci. Methods 89:17-24, 1999; and Takatusgu Komiyama et al., J. Neurosci. Methods 122:195-200, 2003). In a particularly preferred embodiment, the SC medium is_Dulbecco's modified Eagles medium (DEME)supplemented with 10% fetal bovine serum (FBS), 1% penicillin/streptomycin, 152 μg Forskolin and 20 μg/ml Bovine Pituitary Extract (PEX).

In another aspect, the invention provides a conditioned medium for culturing Schwann cells, and the conditioned medium was produced by a method comprising the stpes of (a) providing a culture medium, and cells; (b) culturing the cells in the culture medium, thereby conditioning the medium; and(c) harvesting the conditioned medium from the culture; wherein the cells used to condition the medium are differentiated Ntera2/D1 (NT2)cells.

The conditioned medium of the invention improves the effects of the survival and/or proliferation of Schwann cells and keeps these cells with differentiation capacity.

Definitions

A “Schwann cell” is a cell of neural crest origin that forms a continuous envelope around each peripheral nerves fiber in situ. A Schwann cell can be identified by detecting the presence of one or more markers of Schwann cell such as glial fibrillar acidic protein (GFAP), protein S100, laminin, or nerve growth factor (NGF) receptor, e.g., using antibodies against these markers. Furthermore, Schwann cells have a characteristic morphology that can be detected by microscopic examination of cultures thereof.

A “growth environment” is an environment in which cells of interest will proliferate in vitro. Features of the environment include the medium in which the cells are cultured, the temperature, the partial pressure of O₂ and CO₂.

A “culture medium” is a nutrient solution used for growing cells that typically provides at least one component selected from the group consisting the following categories: amino acids, salts, vitamins, minerals, trace metals, sugars, lipids and nucleosides. Once the culture medium is incubated with cells, it is known to those skilled in the art as “conditioning the medium”. Conditioned medium contains many of the original components of the medium, as well as a variety of cellular metabolites and secreted proteins.

A “NTera2/D1 (NT2) cell” is derived from a human embryonal teratocarcinoma and can be differentiate into neurons while culturing with differentiation agents like hexamethylene bisacetamide (HMBA), retinoic acid or bromodeoxyuridine (BrdUrd).

### EXAMPLES

The following non-limiting examples are provided to further illustrate embodiments of the invention. However, the examples are not intended to be all inclusive and are not intended to limit the scope of the invention described herein.

### Example 1

### Isolation of Schwann Cells

Schwann cells were isolated from rat Sciatic Nerve with the following procedures:

The sciatic nerve tissue of mature SD rat was sliced into 1-3 mm pieces and placed on a 6 cm-culture dish, then these tissues were cultured in 1.5 ml DEME medium supplemented with 10% FBS. Considerable quantities of fibroblasts and Schwann cells are existed in the sciatic nerve tissue, and the separation of these two kinds of cells can be achieved by their different migration rates.

The tissue clusters were transferred to new culture dishes and refresh the medium every week. After about 5 weeks, the remaining cells were harvested after moving out cultured tissue clusters and these cells were rinsed in phosphate buffer saline (Life technologies) for several times.

0.3 ml of Trypsin/EDTA solution (0.25%, Life technologies) was applied to wash the cells and stand for 2 minutes at room temperature. The suspension with cells was replaced into culture dishes that were coated with anti thy 1.1 (1:1000, Sigma) previously, the suspension was cultured for 30 minutes at room temperature. The suspension with un-attached cells was transferred to another culture dish that coated with anti thy 1.1 (1:1000, Sigma) previously. The steps were repeated for four times, after which the suspension was centrifuged for 5 minutes at 1500 rpm. Then, the supernatant was removed and the sediment was re-suspended, whereafter the Schwann cells were cultured in the SC medium consisting of: Dulbecco's modified Eagles medium (DEME, Life technologies) supplemented with 10% fetal bovine serum (FBS, Life Technologies), 1% penicillin/streptomycin (Life Technologies), 152 μg Forskolin (FSK, sigma) and 20 μg/ml Bovine Pituitary Extract (PEX, sigma).

### Example 2

### Preparation of Conditioned Medium

Undifferentiated NTera2/D1 (NT2) cells (about 106 cells) (Stratagene) were obtained from Stratagene (La Jolla, Calif., U.S.A.). To differentiate NTera2/D1 (NT2) cells, cells were cultured at 37° C. in DMEM/F-12 (Dulbecco's modified Eagle's medium/Ham's nutrient mixture F-12) with 10 μM All-trans-Retinoic acid (ATRA 6262, Sigma). The medium was refreshed twice a week. Once obtained, the differentiated NTera2/D1 (NT2) cells were continued culturing for at least 10 days and then the conditioned medium was harvested. Before the Schwann cells were cultured, the conditioned medium was filtrated by 0.22 μm pore sized filter.

### Example 3

### Culture Method of Schwann Cells

Schwann cells purified as described in example 1 were cultured separately in a mixed medium of NT2 conditioned medium/SC medium (mixed in 1:1 ratio) or a SC medium, under the condition of 37° C., 5% CO₂ environment.

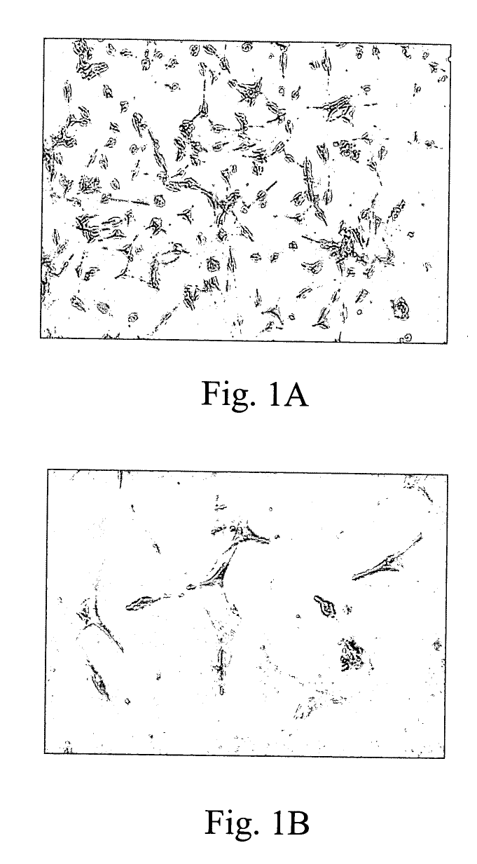

According to the data shown in table 1, the cells in the mixed medium of NT2 conditioned medium/SC medium was survived by 18 days, which is 7 days more than that in SC medium. In addition, the growth condition of Schwann cells cultured separately in NT2 conditioned medium/SC medium and SC medium at the fifteenth day, based on FIG. 1A and FIG. 1B, the cells cultured in NT2 conditioned medium/SC medium still showed normal growth condition after being cultured for 15 days.

TABLE 1

| Medium source | Survival period (day) |
|---|---|
| NT2 conditioned medium/SC medium | 18 |
| SC medium | 11 |

### Example 4

### Evaluation of the Differentiation Ability of Schwann Cells

The differentiation ability of Schwann cells was evaluated with the particular protein S100 and performed with marked S100 antibodies; the marked S100 antibodies were produced as following:

Samples were fixed in 4% Paraformaldehyde (Sigma) for 60 minutes, 0.2% Triton X-100 (Sigma) was applied, and the reaction was set for 5 minutes at room temperature. Non-specific antibodies were blocked with bovine serum albumin, and cultured with monoclonal anti-S100 antibodies (Sigma #S2532) for 60 minutes, then reacted with Fluorescein (FITC)-conjugated AffiniPure Goat Anti-Mouse IgG (H+L) (Jackson ImmunoResearch Laboratories, Inc.) for another 60 minutes. The reaction was then washed in PBS, covered and observed by fluorescence microscope (Leica-090-135.002).

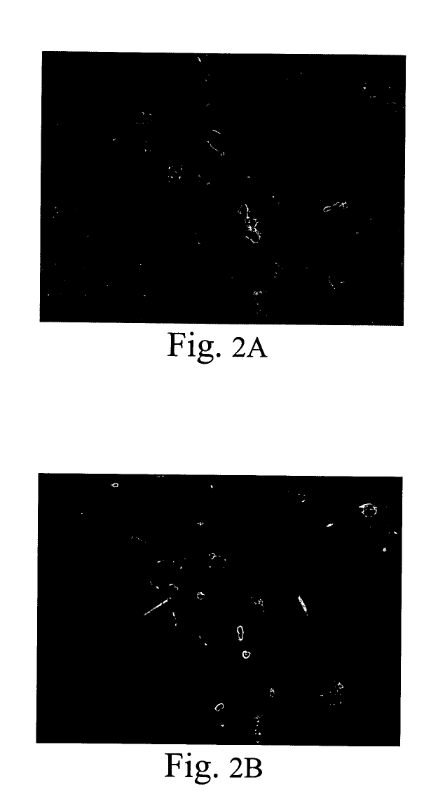

Results are shown in FIG. 2, wherein protein S100 still expressed by Schwann cells that cultured in vitro. FIG. 2A shows the morphology of Schwann cells observed by an Optical microscope; and FIG. 2B shows the green fluorescence expression of protein S100 by Schwann cells cultured in vitro.

Although the invention has been explained in relation to its preferred embodiment, it is to be understood that many other possible modifications and variations can be made without departing from the spirit and scope of the invention as hereinafter claimed.

1. A method for preparing a conditioned medium for culturing Schwann cells, comprising the steps of:
a) providing a culture medium, and cells;
b) culturing the cells in the culture medium, thereby conditioning the medium; and
c) harvesting the conditioned medium from the culture; wherein the cells used to condition the medium are differentiated Ntera2/D1 (NT2)cells.

2. The method of claim 1, wherein the culture medium is DMEM/F12.

3. The method of claim 1, wherein the conditioned medium was produced after differentiated Ntera2/D1 (NT2) cells were cultured for at least 10 days.

4. The method of claim 1,wherein the conditioned medium is filtrated after harvesting or before use.

5. The method of claim 1,wherein the filtration is performed with a 0.22 μm pore sized filter.

6. The method of claim 1, wherein further comprises a step a1), differentiating NTera2/D1 (NT2) cells before step a).

7. The method of claim 6, wherein the NTera2/D1 (NT2) cells are differentiated through incubating undifferentiated NTera2/D1 (NT2) cells in a culture medium containing a differentiation agent in step a).

8. The method of claim 7, wherein the differentiation agent is hexamethylene bisacetamide (HMBA), retinoic acid(RA) or bromodeoxyuridine (BrdUrd).

9. The method of claim 8, wherein the differentiation agent is retinoic acid(RA).

10. The method of claim 8, wherein the differentiation agent is All-trans-Retinoic acid.

11. A method for culturing Schwann cells which comprises stpes of: culturing the Schwann cells in a growth environment that contains a conditioned medium produced by a method comprising steps of:
a) providing a culture medium, and cells;
b) culturing the cells in the culture medium, thereby conditioning the medium; and
c) harvesting the conditioned medium from the culture;
wherein the cells used for the medium conditioning are differentiated Ntera2/D1 (NT2) cells.

12. The method of claim 11, wherein the culture medium is DMEM/F12.

13. The method of claim 1, wherein the conditioned medium was harvested after differentiated Ntera2/D1 (NT2)cells were cultured for at least 10 days.

14. The method of claim 1, wherein the conditioned medium is filtrated after harvesting or before use.

15. The method of claim 11, further comprises a diluting method of the conditioned medium.

16. The method of claim 15, wherein the diluting method comprises steps of:
a) providing a conditioned medium and a diluent medium; and
b) mixing the conditioned medium with the diluent medium.

17. The method of claim 16, wherein the conditioned medium and the diluent medium are mixed in a ratio of 1:1.

18. The method of claim 16, wherein the diluent medium is SC medium.

19. A conditioned medium for culturing Schwann cells, produced by a method comprising:
a) providing a culture medium, and cells
b) culturing the cells in the culture medium, thereby conditioning the medium; and
c) harvesting the conditioned medium from the culture; wherein the cells used to condition the medium are differentiated Ntera2/D1 (NT2)cells.

20. The conditioned medium of claim 19,wherein the culture medium is DMEM/F12.

21. The conditioned medium of claim 19, wherein the conditioned medium was harvested after differentiated Ntera2/D1 (NT2)cells were cultured for at least 10 days.

22. The conditioned medium of claim 19, wherein the conditioned medium is filtrated after harvesting or before use.
